(19) United States
(12) Patent Application Publication
(10) Pub. No.: US 2005/0069900 A1
(43) Pub. Date: Mar. 31, 2005

(54) Analyte sample detection

(75) Inventor: Brian B. Lentrichia, Acton, MA (US)

Correspondence Address: Bingham McCuthen, LLP;Suite 1800, Three Embarcadero, San Francisco, CA, 94111-4067, US

(73) Assignee: Cytyc Corporation, Boxborough, MA (US)

(21) Appl. No.: 10/672,914

(22) Filed: Sep. 25, 2003

(51) Int. Cl. C12Q 1/68; G01N 33/53

(52) U.S. Cl. 435/6; 435/7.1

(57) ABSTRACT

A method for assaying a sample for the presence of a target is provided. The method comprises drawing a liquid sample transversely through a filter using an automated device that can control the rate of flow through the filter. A sensor molecule is attached to the filter which can bind to a target molecule. Binding of the target to the attached sensor molecule can then be detected through any of various techniques. The methods may be performed in multiplex form to permit simultaneous analysis of a plurality of targets. Kits for performing the methods are also provided.

### TECHNICAL FIELD

This invention relates to methods and articles useful in detecting target substances in a sample such as a cytological specimen.

### BACKGROUND OF THE INVENTION

Medical diagnostic testing methods are critical screening tools for the early detection of pathological conditions. Early detection permits the identification of such conditions at a stage when successful treatment is more likely. Early treatment also frequently involves less damaging or less invasive treatment methods and decreases the impact on the patient. In addition to routine screening, diagnostic testing is also used in a variety of other applications, including biopsy analysis and monitoring the results of ongoing medical treatment.

Limitations on the amount of diagnostic information that can be obtained from a sample include the size of the sample that can be obtained and readily manipulated, the processing time required to perform multiple tests, the tolerance of the sample for multiple treatment steps without loss of signal, and the cost for performing multiple methods of analysis. Often it is desirable to obtain additional information on a variety of species which are or may be present in the sample, and/or to obtain information about smaller components such as viruses or other cellular or other species which may occur in a sample.

There is a need in the art for improved procedures for analyzing samples, and for compositions and articles of manufacture useful in such methods.

### SUMMARY OF THE INVENTION

A method for assaying a sample for the presence of a target is provided. The method comprises drawing a liquid sample transversely through a filter using an automated device that can control the rate of flow through the filter. A sensor molecule is attached to the filter which can bind to a target molecule. Binding of the target to the attached sensor molecule can then be detected through any of various techniques. The methods may be performed in multiplex form to permit simultaneous analysis of a plurality of targets. Kits for performing the methods are also provided.

### BRIEF DESCRIPTION OF THE DRAWINGS

FIG. 1 depicts the difference in optical density between the test and control samples with the number of passes of the sample through the filter. An antibody to a Chlamydia antigen was attached to disk filters, and the samples were passed through separate filters in both directions. The filters were then washed and contacted with a second enzyme-labeled antibody. The filters were then placed in a solution of a chromogenic substrate. The optical density reflects the absorbance of the colored product produced by enzymatic conversion of the substrate from the secondary antibody bound to the target complex on the filter.

### DETAILED DESCRIPTION OF THE INVENTION

A sample-conserving method is provided for assaying a sample for a target. A liquid sample is drawn transversely through a filter using an automated device that can control the rate of flow through the filter. A sensor molecule is attached to the filter which can bind to a target molecule. If target is present in the sample, it binds to the sensor and is retained on the filter. Binding of the target can then be detected through any of various techniques, including sandwich techniques and competitive binding methods. The methods can be used in multiplex form.

By using a thin layer of filter material with a relatively high binding capacity, the method conserves the sample volume, which can be recovered for performing additional tests. In one aspect, the entire contents of a liquid sample can be cycled through the filter, and then recovered for use in additional analyses. The sample may be returned to its original container, and may be passed back through the filter to accomplish this. The methods described herein can provide improved sensitivity as compared to microtiter EIA. The filter can be disposable which can eliminate carry over between samples, lowering the risk of cross-contamination. The automated method advantageously does not require handling-intensive steps such as pipetting or centrifugation.

The methods and apparatus disclosed herein can be applied to a number of prognostic markers including analytes for cancer progression, metastatic potential, reoccurrence after treatment, response to chemotherapy and immediate precancerous conditions in breast, ovary, endometrium, colon, cervix, prostate, lung, liver, blood, bone or nervous system.

The methods can provide improved techniques for target detection in a sample. For example, sensitivity in enzyme immunoassay is governed by the sample size and the amount of antibody contacting the sample. Current EIA microtiter well formats immobilize between 2 and 5 micrograms of antibody per well (i.e, per test), and can handle an assay volume of 0.1 mL. A filter of the size used in the ThinPrep 2000 ® Processor when derivatized with antibody as described herein can immobilize over 100 fold more antibody than a microtiter plate well and can be contacted With a much larger sample volume than available in microtiter assays. Combined with the additional sensitivity of using a chemiluminescent substrate, the increase in sensitivity over traditional microtiter ELISA can be 500,000 to 1,000,000 fold. This can allow the detection of less than 10,000 molecules of target protein. Use of the apparatus advantageously allows for automated temperature control; increasing the temperature of the reactants can double the speed of the reaction with every 10 degrees of increase.

The sample(s) can be processed by a suitable apparatus and the filters analyzed by an automated imaging system. The methods can be incorporated into sample processing devices such as those described in U.S. Pats. Nos. 5,185,084, 5,266,495, 6,010,909, 6,225,125, and 5,942,700, all assigned to Cytyc Corp. Such an adapted device allows for a binding assay using precisely metered volumes of fluid by controlling flow through the filter. A color in produced in solution in the presence of target from an enzymatic product such as a chemiluminescent stain can be detected through any method known or discoverable in the art, including densitometry of the solution.

In one variation, the sample can be subjected to the binding assay after withdrawal of a portion of that sample for other test(s). For example, the suspension of exfoliated cells remaining in a specimen vial after slide preparation using automated processors such as the ThinPrep 2000® offers the opportunity for molecular testing.

In another variation, microparticles with a sensor molecule attached to their surface can be added to the sample to first capture the target on the particles. The particles themselves can then be collected on a filter (either inert or activated, which may be derivatized with other molecules that bind to the sensor and/or target and/or a component of the particles), and the amount of target analyte bound to the particles can be determined on the filter after contacting it with a detecting reagent, either on the surface of the filter or after depositing the particles into a solution containing the detection reagent. Exemplary types of particles which may be used include magnetic particles, polystyrene latex particles, and shell type particles.

Before the present invention is described in further detail, it is to be understood that this invention is not limited to the particular methodology, solutions or apparatuses described, as such methods, solutions or apparatuses can, of course, vary. It is also to be understood that the terminology used herein is for the purpose of describing particular embodiments only, and is not intended to limit the scope of the present invention.

Use of the singular forms “a,” “an,” and “the” include plural references unless the context clearly dictates otherwise. Thus, for example, reference to “a sample” includes a plurality of samples, reference to “a sensor” includes a plurality of such sensors, reference to “a target” includes a plurality of targets, and the like. Additionally, use of specific plural references, such as “two,” “three,” etc., read on larger numbers of the same subject unless the context clearly dictates otherwise.

Terms such as “connected,” “attached,” and “linked” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each sub-combination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise or the context clearly dictates otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications mentioned herein are hereby incorporated by reference for the purpose of disclosing and describing the particular materials and methodologies for which the reference was cited. The publications discussed herein are provided solely for their disclosure prior to the filing date of the present application. Nothing herein is to be construed as an admission that the invention is not entitled to antedate such disclosure by virtue of prior invention.

The terms “polynucleotide,” “oligonucleotide,” “nucleic acid” and “nucleic acid molecule” are used interchangeably herein to refer to a polymeric form of nucleotides of any length, and may comprise ribonucleotides, deoxyribonucleotides, analogs thereof, or mixtures thereof. These terms refer only to the primary structure of the molecule. Thus, the terms includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

Suitable hybridization conditions for a given assay format can be determined by one of skill in the art; nonlimiting parameters which may be adjusted include concentrations of assay components, pH, salts used and their concentration, ionic strength, temperature, etc.

More particularly, the terms “polynucleotide,” “oligonucleotide,” “nucleic acid” and “nucleic acid molecule” include polydeoxyribonucleotides (containing 2-deoxy-D-ribose), polyribonucleoti des (containing D-ribose), including tRNA, rRNA, hRNA, and mRNA, whether spliced or unspliced, any other type of polynucleotide which is an N- or C-glycoside of a purine or pyrimidine base, and other polymers containing alternative backbones, including peptide nucleic acid (PNA) and linked nucleic acid LNA, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA; as well as double- and single-stranded RNA, and hybrids thereof including for example hybrids between DNA and/or RNA and/or PNA and/or other forms, and also include known types of modifications, for example, labels, alkylation, “caps,” substitution of one or more of the nucleotides with an analog, internucleotide modifications such as, for example, those with negatively charged linkages (e.g., phosphorothioates, phosphorodithioates, etc.), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base which form hydrogen bonds to a respective complementary pyrimidine or purine. The resultant modified nucleotidic unit optionally may form a base pair with other such modified nucleotidic units but not with A, T, C, G or U. Abasic sites may be incorporated which do not prevent the function of the polynucleotide; preferably the polynucleotide does not comprise abasic sites. Some or all of the residues in the polynucleotide can optionally be modified in one or more ways.

Exemplary modified nucleotidic units include aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, 2,6-diaminopurine, and “locked” nucleic acid units (LNA) and an analog thereof. Koshkin, et al., Tetrahedron Letters 1998 39:4381-4384; PCT Publ. No. WO99/14226.

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′-H and C6-oxy, respectively, of guanosine. Thus, for example, guanosine (2-amino-6-oxy-9-β-D-ribofuranosyl-purine) may be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described in U.S. Pat. No. 5,780,610 to Collins et al. Other normatural base pairs may be synthesized by the method described in Piccirilli et al. (1990) Nature 343:33-37 for the synthesis of 2,6-diaminopyrimidine and its complement (1-methylpyrazolo-[4,3]pyrimidine-5,7-(4H,6H)-dione). Other such modified nucleotidic units which form unique base pairs are known, such as those described in Leach et al. (1992) J. Am. Chem. Soc. 114:3675-3683 and Switzer et al., supra.

“Complementary” or “substantially complementary” refers to the ability to hybridize or base pair between nucleotides or nucleic acids, such as, for instance, between a sensor peptide nucleic acid and a target polynucleotide. Complementary nucleotides are, generally, A and T (or A and U), or C and G. Two single-stranded polynucleotides or PNAs are said to be substantially complementary when the bases of one strand, optimally aligned and compared and with appropriate insertions or deletions, pair with at least about 80% of the bases of the other strand, usually at least about 90% to 95%, and more preferably from about 98 to 100%.

Alternatively, substantial complementarity exists when a polynucleotide or PNA will hybridize under selective hybridization conditions to its complement. Typically, selective hybridization will occur when there is at least about 65% complementary over a stretch of at least 14 to 25 bases, preferably at least about 75%, more preferably at least about 90% complementary. See, M. Kanehisa Nucleic Acids Res. 12:203 (1984).

“Preferential binding” or “preferential hybridization” refers to the increased propensity of one polynucleotide or PNA to bind to its complement in a sample as compared to a noncomplementary polymer in the sample.

Hybridization conditions will typically include salt concentrations of less than about IM, more usually less than about 500 mM and preferably less than about 200 mM. In the case of hybridization between a peptide nucleic acid or other similar nucleic acid and a polynucleotide, the hybridization can be done in solutions containing little or no salt. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art.

The terms “aptamer” (or “nucleic acid antibody”) is used herein to refer to a single- or double-stranded polynucleotide that recognizes and binds to a desired target molecule by virtue of its shape. See, e.g., PCT Publication Nos. WO 92/14843, WO 91/19813, and WO 92/05285.

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include polypeptides contain [post-translational] modifications of the polypeptide, for example, glycosylations, acetylations, phosphorylations, and sulphations. In addition, protein fragments, analogs (including amino acids not encoded by the genetic code, e.g. homocysteine, ornithine, D-amino acids, and creatine), natural or artificial mutants or variants or combinations thereof, fusion proteins, derivatized residues (e.g. alkylation of amine groups, acetylations or esterifications of carboxyl groups) and the like are included within the meaning of polypeptide.

As used herein, the term “binding pair” refers to first and second molecules that bind specifically to each other with greater affinity than to other components in the sample. The binding between the members of the binding pair is typically noncovalent. Exemplary binding pairs include immunological binding pairs (e.g. any haptenic or antigenic compound in combination with a corresponding antibody or binding portion or fragment thereof, for example digoxigenin and anti-digoxigenin, fluorescein and anti-fluorescein, dinitrophenol and anti-dinitrophenol, bromodeoxyuridine and anti-bromodeoxyuridine, mouse immunoglobulin and goat anti-mouse immunoglobulin) and nonimmunological binding pairs (e.g., biotin-avidin, biotin-streptavidin, hormone [e.g., thyroxine and cortisol]-hormone binding protein, receptor-receptor agonist or antagonist (e.g., acetylcholine receptor-acetylcholine or an analog thereof), IgG-protein A, lectin-carbohydrate, enzyme-enzyme cofactor, enzyme-enzyme-inhibitor, and complementary polynucleotide pairs capable of forming nucleic acid duplexes) and the like. One or both member of the binding pair can be conjugated to additional molecules.

The term “antibody” as used herein includes antibodies obtained from both polyclonal and monoclonal preparations, as well as: hybrid (chimeric) antibody molecules (see, for example, Winter et al: (1991) Nature 349:293-299; and U.S. Pat. No. 4,816,567); F(ab′)₂ and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human hybridomas or from murine hybridomas made from mice expression human immunoglobulin chain genes or portions thereof. See, e.g., Cote, et al. Monoclonal Antibodies and Cancer Therapy, Alan R. Liss, 1985, p. 77.

“Multiplexing” herein refers to an assay or other analytical method in which multiple analytes can be assayed simultaneously.

“Optional” or “optionally” means that the subsequently described event or circumstance may or may not occur, and that the description includes instances where the event or circumstance occurs and instances in which it does not.

The Sample

The sample to be analyzed can be any source of biological material that can be obtained from a living organism directly or indirectly, including cells, tissue or fluid. Nonlimiting examples of the sample include blood, urine, semen, milk, sputum, mucus, plueral fluid, pelvic fluid, sinovial fluid, ascites fluid, body cavity washes, eye brushing, skin scrapings, a buccal swab, a vaginal swab, a pap smear, a rectal swab, an aspirate, a needle biopsy, a section of tissue obtained for example by surgery or autopsy, plasma, serum, spinal fluid, lymph fluid, the external secretions of the skin, respiratory, intestinal, and genitourinary tracts, tears, saliva, tumors, organs, a microbial culture, a virus, and samples of in vitro cell culture constituents.

The sample can be a positive control sample which is known to contain the target. A negative control sample can also be used which is used to determine whether a given set of conditions produces false positives.

The sample can be collected or placed in a solution used for liquid based cytology or a medium that lyses the cells and dissolves all of the molecular components into solution. In one embodiment, the sample may comprise a preservative solution such as PreservCyt® Solution (Cytyc Corp.).

The sample can comprise a preservative solution suitable for preservation of cells and tissue at ambient temperatures. The solution can comprise an alcohol and preferably a buffer, and can be used for in vitro preservation of mammalian cells at ambient temperatures following biopsy, and prior to staining or other forms of analysis. The solution can be one such as described in U.S. Pat. No. 5,256,571 to Hurley et al. issued Oct. 26, 1993. The preservative solution can comprise a water-miscible alcohol, and preferably an anti-clumping agent and a buffering agent. The alcohol constituent is present in an amount sufficient to fix sample cells or tissue while still permitting acceptable binding of the sensor to its target. The alcohol is typically a lower alkyl (C₁₋₆) alcohol, and may be a C₁₋₄ alcohol, and may be selected from the group consisting of methanol, ethanol and isopropanol. The alcohol may be present in an amount greater than about 40% and less than about 60%, and may be about 45% or more, and may be about 55% or less. In another variation, the alcohol is present in an amount of at least approximately 20 percent by solution. The anti-clumping agent may be present in an amount sufficient to prevent cells from clumping in solution. Any suitable anti-clumping agent effect in the alcoholic preservative solution can be used, and can be, for example, a chelating agent selected, for example, from the group consisting of ethylenediaminetetra-acetic acid (EDTA), and its salts, such as disodium, tripotassium and tetrasodium. Other agents deemed useful as the anti-clumping agent include cuminin, heparin, streptokinase, and such agents found in lysing or anticoagulant compositions. Any buffering agent which can maintain the preservative solution at a desired pH during use may be used. Exemplary buffering agents include PBS, Tris, sodium acetate, and citric acid. EDTA and its salts may also be used as a buffering agent. The buffering agent can be one which maintains the pH of the solution within a range of between about four to about seven for the duration of preservation. Accordingly, a preferred buffer is an acetate buffer, such as sodium acetate, magnesium acetate, calcium acetate, and combinations thereof.

A detergent may also be used in one or more of the liquids used in the methods, including in the sample. The detergent may be non-ionic, cationic, anionic or zwitterionic. Mixtures of detergents may also be used. Exemplary classes of detergents include alcohol ether sulfates, alcohol sulfates, alkanolamides, alkyl sulfonates, amine oxides, amphoteric detergents, anionic detergents, betaine derivatives, cationic detergents, disulfonates, dodecylbenzene sulfonic acid, ethoxylated alcohols, ethoxylated alkyl phenols, ethoxylated fatty acids, glycerol esters hydrotropes, lauryl sulfates, mono and diglycerides, non-ionic detergents, phosphate esters, quaternary detergents, and sorbitan derivatives.

The Target

The target may be any component of the sample that is desired to be detected. Nonlimiting examples of the target include a polynucleotide, a protein, a peptide, a polysaccharide, mucopolysaccharide, proteoglycan, a carbohydrate, a lipid, a fat, a cell, a cell type, an organism, a virus, a structure, an antigen, an inorganic compound, or other molecule to which a sensor can be obtained.

Exemplary molecular targets include HPV E2 protein, HPV E6 and E7 proteins, HPV L1 capsid protein, p16INK4a, E-cadherin, N-cadherin, p53, GCDFP-15, Pericyclin, NuMA, carbonic anhydrase, matrix metalloproteinases, nuclear matrix proteins, ferritin, aurora A, pericentrin, osteopontin, prostatin, insulin-like growth factor, fibroblast growth factor, BRCA1, BRCA2, mammoglobin, PSE, CEA, CA-125, CA 19-9, CA 15-3, somatostatin, synaptophysin, chromogranin, kallikriens, fibronectin, EGFR, K-ras, Her-2/neu, treponemal antigen, neuron-specific enolase, retinoblastoma protein, hepatitis C surface antigen, sexually transmitted disease markers including the outer membrane protein of Chlamydia trachomatis, cancer markers, and HIV.gp120.

Exemplary viral targets include: any of the herpes viruses including cytomegalovirus, HSV-1, and HSV-2; any of the papillomaviruses, including those associated with diseases including cervical cancer, including types 16, 18, 31, 33, 35, 39, 45, 51, 52, 53, 56, 58, 59, 66, 68 and 70; any of the lentiviruses, including HIV-1, HIV-2, feline leukemia virus; SARS; rubella; West Nile Virus; Epstein Barr virus, adenovirus, and any of the subtypes of any thereof.

Where the target is a cell or cell component or product, the cell can be of any origin, including prokaryotic, eukaryotic, or archea. The cell may be living or dead. If obtained from a multicellular organism, the cell may be of any cell type. The cell may be a cultured cell line or a primary isolate, the cell may be mammalian, amphibian, reptilian, plant, yeast, bacterial, mycobacterial, spirochetal, or protozoan. The cell may be human, murine, rat, hamster, chicken, quail, or dog. The cell may be a normal cell, a mutated cell, a genetically manipulated cell, a tumor cell, etc.

Exemplary cell types from multicellular organisms include acidophils, acinar cells, pinealocytes, adipocytes, ameloblasts, astrocytes, basal (stem) cells, basophils, hepatocytes, neurons, bulging surface cells, C cells, cardiac muscle cells, centroacinar cells, chief cells, chondrocytes, Clara cells, columnar epithelial cells, corpus luteal cells, decidual cells, dendrites, endrocrine cells, endothelial cells, enteroendocrine cells, eosinophils, erythrocytes, extraglomerular mesangial cells, fetal fibroblasts, fetal red blood cells, fibroblasts, follicular cells, ganglion cells, giant Betz cells, goblet cells, hair cells, inner hair cells, type I hair cells, hepatocytes, endothelial cells, Leydig cells, lipocytes, liver parenchymal cells, lymphocytes, lysozyme secreting cells, macrophages, mast cells, megakaryocytes, melanocytes, mesangial cells, monocytes, myoepithelial cells, myoid cells, neck mucous cells, nerve cells, neutrophils, oligodendrocytes, oocytes, osteoblasts, osteochondroclasts, osteoclasts, osteocytes, pillar cells, sulcal cells, parathyroid cells, parietal cells, pepsinogen-secreting cells, pericytes, pinealocytes, pituicytes, plasma cells, platelets, podocytes, spermatocytes, Purkinje cells, pyramidal cells, red blood cells, reticulocytes, Schwann cells, Sertoli cells, columnar cells, skeletal muscle cells, smooth muscle cells, somatostatin cells, enteroendocrine cells, spermatids, spermatogonias, spermatozoas, stellate cells, supporting Deiter cells, support Hansen cells, surface cells, surface epithelial cells, surface mucous cells, sweat gland cells, T lymphocytes, theca lutein cells, thymocytes, thymus epithelial cell, thyroid cells, transitional epithelial cells, type I pneumonocytes, and type II pneumonocytes.

Exemplary types of tumor cells include adenomas, carcinomas, adenocarcinomas, fibroadenomas, ameloblastomas, astrocytomas, mesotheliomas, cholangiocarcinomas, cholangiofibromas, cholangiomas, chondromas, chondrosarcomas, chordomas, choriocarcinomas, craniopharyngiomas, cystadenocarcinomas, cystadenomas, dysgerminomas, ependymomas, epitheliomas, erythroid leukemias, fibroadenomas, fibromas, fibrosarcomas, gangliogliomas, ganglioneuromas, ganglioneuroblastomas, gliomas, granulocytic leukemias, hemangiomas, hemangiopericytomas, hemangiosarcomas, hibernomas, histiocytomas, keratoacanthomas, leiomyomas, leiomyosarcomas, lipomas, liposarcomas, luteomas, lymphangiomas, lymphangiosarcomas, lymphomas, medulloblastomas, melanomas, meningiomas, mesotheliomas, myelolipomas, nephroblastomas, neuroblastomas, neuromyoblastomas, odontomas, oligodendrogliomas, osteochondromas, osteomas, osteosarcomas, papillomas, paragangliomas, pheochromocytomas, pinealomas, pituicytomas, retinoblastomas, rhabdomyosarcomas, sarcomas, schwannomas, seminomas, teratomas, thecomas and thymomas.

Exemplary bacteria include Staphylococcus aureus, Legionella pneumophila, Escherichia coli, M. tuberculosis, S. typhimurium, Vibrio cholera, Clostridium perfringens, Clostridium tetani, Clostridium botulinum, Clostridium baratii, Clostridium difficile, M. leprae, Gardnerella vaginalis, Helicobacter pylori, Hemophilus influenzae type b, Corynebacterium diphtheriae, Corynebacterium minutissimum, Bordetella pertussis, Streptococcus pneumoniae, Neisseria gonorrhoeae, Neisseria meningitides, Shigella dysenteriae, Pseudomonas aeruginosa, Bacteroides fragilis, Prevotella melaninogenica, Fusobacterium, Erysipelothrix rhusiopathiae, Listeria monocytogenes, Bacillus anthracis, Hemophilus ducreyi, Francisella tularensis, Yersinia pestis, Bartonella henselae, Klebsiella, Enterobacter, Serratia, Proteus, and Shigella.

Exemplary spirochetes include Treponema pallidum, T. pertenue, T. carateum, Borrelia recurrentis, B. vincentii, B. burgdorferi, and Leptospira icterohaemorrhagiae.

Exemplary fungi include Actinomyces bovis, Actinomyces israelii, Aspergillus fumigatus, Blastomyces dermatitidis, Candida albicans, Coccidioides immitis, Cryptococcus neoformans, Histoplasma capsulatum, Nocardia asteroides, Pneumocystis carinii, Sporothrix schenckii, Sporotrichum schenckii, Pichia pastoris, Saccharomyces cerevisiae, and Schizosaccharomyces pombe.

Exemplary protozoa and parasites which include Plasmodium falciparum, Entamoeba histolytica, trypansomes, Leishmania, Toxpolasma gondii, Trichomonas, Giardia lamblia, and Chlamydia trachomatis (including the elementary body).

Where the target is a polynucleotide, the target polynucleotide can be single-stranded, double-stranded, or higher order, and can take any topology, for example linear, circular, branched. Exemplary single-stranded target polynucleotides include mRNA, rRNA, tRNA, hnRNA, ssRNA or ssDNA viral genomes, although these polynucleotides may contain internally complementary sequences and significant secondary structure. Exemplary double-stranded target polynucleotides include genomic DNA, mitochondrial DNA, chloroplast DNA, dsRNA or dsDNA viral genomes, plasmids, phage, and viroids.

The Sensor

The sensor can be any substance which can selectively bind to its target when presented on the filter and contacted by the sample. Nonlimiting examples of the sensor include a polynucleotide as described above, including a peptide nucleic acid and an aptamer, and an antibody as described above. Combinations of different sensors may also be used, which can allow for the detection and analysis of a plurality of targets in the sample. In one variation, the sensor may be a PNA that binds specifically to a target polynucleotide suspected of being present in the sample.

In another variation, the sensor may be an aptamer. Preparation of oligonucleotides which bind to a desired target has been described by Blackwell, T. K., et al., Science (1990) 250:1104-1110; Blackwell, T. K., et al., Science (1990) 250:1149-1152; Tuerk, C., and Gold, L., Science (1990) 249:505-510; Joyce, G. F., Gene (1989) 82:83-87; U.S. Pat. No. 5,270,163 to Gold et al. issued Dec. 14, 1993.

Multiplex detection can be accomplished in any available manner. For example, different sensors specific for different targets may be bound to particular locations on the filter, which locations can then be interrogated for binding of target. Different labels may be used on different secondary sensors specific for different targets which allow for multiplex detection. Techniques for detecting such binding are known in the art; a number of discrete imaging systems are commercially available, including Cytyc Corporation's ThinPrep® Imaging System, the TriPath FocalPoint™ Profiler, the ChromaVision Acis® System, the CompuCyt iCyte Imaging System, the Applied Imaging CytoVision™ System, and the Veracel Verasys Imaging System. Such an apparatus can be modified to incorporate steps for performing an additional assay on a sample and/or to incorporate one or more detection systems for the additional label(s) used to detect target(s). Alternatively, the filter can be imaged with a CCD camera and the image digitized for quantification.

Sandwich techniques may be used to detect binding of the target to the sensor. For example, where the sensor is an antibody specific for a target, a second labeled antibody which does not interfere with the binding of the sensor antibody may be used to allow detection of binding of the target. Similarly, a polynucleotide that binds to a portion of the target or of the sensor:target complex with disrupting such complex can also be used.

The Filter

The filter can be any material to which a sensor can be attached and which does not adversely impact the sample for its other intended uses, and may comprise a plurality of different materials. Exemplary materials include polyester, cellulose, polycarbonate, nylon, and teflon, as described in U.S. Pat. No. 5,942,700 to Cytyc Corp. The filter has pore sizes suitable for allowing the desired portion of the sample to pass through the filter. For example, smaller pore sizes can be used for soluble target species, whereas larger pore sizes may be suitable where the target is a membrane-bound molecule and it is desirable to allow the membrane or cell or organelle comprising the target to pass through the pores to allow maximum binding to all surfaces of the filter. Typically the pore sizes of the filter fall within the range of about 0.2 to about 20 microns where the target is a soluble molecule and it is not desired to pass cells in the sample through the filter. In one variation, the pore size may be selected to permit particular cell types to pass through the filter and be retained on the opposite side. This can be desirable where a subpopulation of cells negatively affects an assay to be performed on the sample. For example, inflammatory cells, which can interfere with cytological interpretation, can pass through the pores of such filters, while the cells or cytological interest are retained on the sample side of the filter. Antibodies specific for cell types desired to be retained on the filter and/or its opposite side can be attached to the filter. The filter is attached to an apparatus which allows the sample to be drawn through the filter. In one embodiment, the filter is attached to a tube or cylinder which can be detachably connected to a pressure-controlling device such as an automated specimen processor. The tube or cylinder may be formed from an alcohol-resistant plastic.

Labels

Labels useful in the inventions described herein include any substance which can be detected, directly or indirectly, in association with target present in the sample upon binding of the sensor to the target.

A secondary sensor molecule attached to a label may be used to detect binding of the target to the sensor on the filter. The secondary sensor molecule can be any species that can bind to the target molecule and/or the target:sensor complex without disrupting such complexes. Typically the secondary sensor molecule is an antibody or a polynucleotide.

Any effective detection method can be used for interrogating the filter, and/or a solution that has contacted the filter, for the presence of label, including optical, spectroscopic, electrical, piezoelectrical, magnetic, Raman scattering, surface plasmon resonance, radiographic, colorimetric, calorimetric, etc. Preferably the label is, can be rendered or can produce a substance that is optically detectable to a human and/or a detection device.

Exemplary labels include a chromophore, a lumiphore, a fluorophore, a chromogen, a hapten, an antigen, a radioactive isotope, a magnetic particle, a metal nanoparticle such as a gold or silver nanoparticle, an enzyme, and one member of a binding pair.

A fluorophore can be any substance which absorbs light of one wavelength and emits light of a different wavelength. Typical fluorophores include fluorescent dyes, semiconductor nanocrystals, lanthanide chelates, and a green fluorescent protein.

Exemplary fluorescent dyes include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2®, Cy3®, Cy3.5®, CyS®, Cy5.5®, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oreg. Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY® FL, BODIPY® FL-Br₂, BODIPY® 530/550, BODIPY® 558/568, BODIPY® 564/570, BODIPY® 576/589, BODIPY® 581/591, BODIPY® 630/650, BODIPY® 650/665, BODIPY® R6G, BODIPY® TMR, BODIPY® TR, conjugates thereof, and combinations thereof. Exemplary lanthanide chelates include europium chelates, terbium chelates and samarium chelates.

A wide variety of fluorescent semiconductor nanocrystals (“SCNCs”) are known in the art; methods of producing and utilizing semiconductor nanocrystals are described in: PCT Publ. No. WO 99/26299 published May 27, 1999, inventors Bawendi et al.; U.S. Pat. No. 5,990,479 issued Nov. 23, 1999 to Weiss et al.; and Bruchez et al., Science 281:2013, 1998. Semiconductor nanocrystals can be obtained with very narrow emission bands with well-defined peak emission wavelengths, allowing for a large number of different SCNCs to be used as signaling chromophores in the same assay, optionally in combination with other non-SCNC types of signaling chromophores.

The term “green fluorescent protein” refers to both native Aequorea green fluorescent protein and mutated versions that have been identified as exhibiting altered fluorescence characteristics, including altered excitation and emission maxima, as well as excitation and emission spectra of different shapes (Delagrave, S. et al. (1995) Bio/Technology 13:151-154; Heim, R. et al. (1994) Proc. Natl. Acad. Sci. USA 91:12501-12504; Heim, R. et al. (1995) Nature 373:663-664). Delgrave et al. isolated mutants of cloned Aequorea victoria GFP that had red-shifted excitation spectra. Bio/Technology 13:151-154 (1995). Heim, R. et al. reported a mutant (Tyr66 to His) having a blue fluorescence (Proc. Natl. Acad. Sci. (1994) USA 91:12501-12504).

Exemplary enzymes include alkaline phosphatase, horseradish peroxidase, β-galactosidase, glucose oxidase, a bacterial luciferase, an insect luciferase and sea pansy luciferase (Renilla koellikeri), which can create a detectable signal in the presence of suitable substrates and assay conditions, known in the art. The enzyme preferably produces a detectable product from the substrate, such as a colored product, a fluorescent product, or a chemiluminescent product.

Exemplary haptens and/or members of a binding pair include avidin, streptavidin, digoxigenin, biotin, and those described above.

Kits

Kits comprising reagents useful for performing the methods of the invention are also provided. In one embodiment, a kit comprises a filter comprising an attached sensor molecule specific for a desired target. A labeled secondary sensor molecule suitable for binding to the target:sensor complex formed on the filter is also provided. The secondary sensor may be conjugated to a label, which may be a chromophore, including a fluorophore. Where the label requires one or more additional reactants, those reactants may be provided. For example, when the label is an enzyme it may require a substrate to produce a detectable signal, which can be included in the kit.

The components of the kit may be retained by a housing. Instructions for using the kit to perform a method of the invention may be provided with the kit, and can be provided in any fixed medium. The instructions may be located inside the housing or outside the housing, and may be printed on the interior or exterior of any surface forming the housing which renders the instructions legible. The kit may be in multiplex form, containing filters with pluralities of one or more different sensors which can bind to corresponding different targets in the sample, and different secondary labeled sensor molecules.

### EXAMPLES

The following examples are set forth so as to provide those of ordinary skill in the art with a complete description of how to make and use the present invention, and are not intended to limit the scope of what is regarded as the invention. Efforts have been made to ensure accuracy with respect to numbers used (e.g., amounts, temperature, etc.) but some experimental error and deviation should be accounted for. Unless otherwise indicated, parts are parts by weight, temperature is degree centigrade and pressure is at or near atmospheric, and all materials are commercially available.

### Example 1

A chemically activated polyester fiber matrix filter with a 5 um nominal pore size is bonded onto a plastic cylinder. Antibody is immobilized covalently onto the filter using a standard one-step room temperature process to create an ‘Immunofilter’. Immobilization of the antibody is irreversible, instantaneous, and requires no special skills or equipment. The Immunofilter is employed with a ThinPrep Processor, or similar device, to affect the remaining steps of the process. The steps of the process are:

Target Antigen Capture. The antibody filter is submerged into a vial containing a specimen collected into PreservCyt® Solution. The solution is passed back and forth through the filter 3-5 times. Antigen in the solution binds to the antigen specific antibody immobilized on the filter. Contents of the filter cylinder are evacuated back into the vial through the filter. The filter and cylinder are removed from the original vial and the remaining steps are performed in one or more separate containers.

Filter Washing. Residual PreservCyt and/or cellular debris and unbound antigen is rinsed from filter with a surfactant-containing buffer. The filter is then contacted with a second antibody linked to a peroxidase enzyme to form an antibody:antigen:antibody-enzyme complex on the filter surface where the antigen target has bound to the filter. Residual second antibody is then rinsed from the filter with buffer by passing the rinse buffer back and forth across the filter.

Detection. A solution is then drawn back and forth across the filter that contains a chemical that is a substrate for the enzyme. The substrate, when acted on by the enzyme, generates an optically detectable product. Generation of the product is monitored by optical absorbance. The rate of formation of the product is measured. The end point absorbance is measured. The increase in optical absorbance is proportional to the amount of antigen present.

### Example 2

Immunodyne ABC™ filters (Pall Corporation) are composed of chemically activated nylon 66 overlaid onto polyester fiber. They are available off the shelf in nominal pore sizes of 0.45, 1.0, 3.0 and 5.0 micron. These filters are compatible with the TP 2000. A monoclonal anti-Chlamydia antibody directed against the Major Outer Membrane Protein was immobilized onto the filter and then the Immunofilter used in experiments with confirmed Chlamydia infected residual specimens that had been collected for the ThinPrep Pap Test. The samples were confirmed to be positive or negative for Chlamydia by DNA amplification methods and immunofluorescence. A sequence was written for the TP 2000 to control the fluid flow and the subsequent sequence of events. A second rabbit derived polyclonal antibody obtained from Biogenesis Inc., Brentwood, N.H., conjugated to a peroxidase enzyme in solution was to contacted with the filter according to the configuration described above and in the attached diagram. After a washing step to remove excess conjugate, the Immunofilter was bathed with a chromogenic substrate (K-blue; Kirkegaard & Perry Laboratories, Gaithersburg, Md.) on the TP2000. The level of blue color appearing in the solution was determined by spectrophotometry.

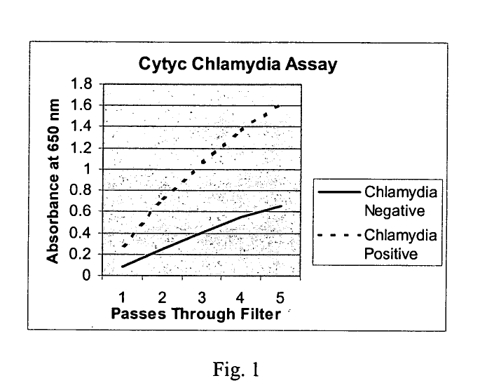

Results. FIG. 1 depicts the difference in optical density between the test and control specimens with the number of passes of the specimen through the filter. The optical density reflects the absorbance of the colored product produced by enzymatic conversion of the substrate from the secondary antibody bound to the target complex on the filter. Control experiments indicated an insignificant increase in optical density with each pass when filters lacking bound antibody (and blocked with bovine serum albumin) were used.

Although the invention has been described in some detail with reference to the preferred embodiments, those of skill in the art will realize, in light of the teachings herein, that certain changes and modifications can be made without departing from the spirit and scope of the invention. Accordingly, the invention is limited only by the claims.

1. A method for assaying a sample for the presence of a target molecule comprising:
providing a liquid sample suspected of comprising the target molecule;
contacting the sample with a filter, said filter comprising a sensor molecule attached thereto, said sensor molecule capable of specifically binding to the target molecule, if present;
passing the sample transversely through said filter using a pressure-controlling apparatus under conditions that allow the sensor molecule to bind to the target molecule;
recovering the remaining liquid sample; and
determining whether the target has bound to the sensor.

2. The method of claim 1, wherein the sample is selected from the group consisting of blood; urine; semen; milk; sputum; mucus; plueral fluid; pelvic fluid, sinovial fluid; ascites fluid; a body cavity wash; eye brushing; skin scrapings; a buccal swab; a vaginal swab; a pap smear; a rectal swab; an aspirate; a needle biopsy; a section of tissue; plasma; serum; spinal fluid; lymph fluid; an external secretion of the skin, respiratory, intestinal, or genitourinary tract; tears; saliva; a tumor; an organ; a microbial culture; and an in vitro cell culture constituent.

3. The method of claim 1, wherein the sensor comprises an antibody.

4. The method of claim 1, wherein the sensor comprises a polynucleotide.

5. The method of claim 1, wherein the sensor comprises a peptide nucleic acid.

6. The method of claim 1, wherein a plurality of different sensors are attached to the filter, wherein each of said plurality can selectively bind to a corresponding different target.

7. The method of claim 1, wherein the target is a cell surface molecule.

8. The method of claim 1, wherein the target is a soluble molecule.

9. The method of claim 1, wherein the target is membrane-bound.

10. The method of claim 1, wherein the target is DNA.

11. The method of claim 1, wherein the target is RNA.

12. The method of claim 1, wherein the target is from a pathological organism.

13. The method of claim 1, wherein the target is a viral marker.

14. The method of claim 1, further comprising comparing a result from said determining to a result obtained from a control sample.

15. The method of claim 14, where the control sample is a positive control.

16. The method of claim 14, where the control sample is a negative control.

17. The method of claim 1, further comprising washing said sample prior to said determining.

18. The method of claim 1, wherein the sample comprises a water-soluble alcohol in an amount effective to preserve the sterility of the solution toward at least one contaminant.

19. The method of claim 1, wherein determining whether the target has bound to the sensor comprises contacting the filter with a labeled secondary sensor, and determining whether label is associated with the filter.

20. The method of claim 19, wherein the first label comprises an agent selected from a chromophore, a lumiphore, a fluorophore, a chromogen, a hapten, an antigen, a radioactive isotope, a magnetic particle, a metal nanoparticle, an enzyme, an antibody or binding portion or equivalent thereof, an aptamer, and one member of a binding pair.

21. The method of claim 20, wherein the agent is an enzyme selected from alkaline phosphatase, horseradish peroxidase, β-galactosidase, glucose oxidase, a bacterial luciferase, an insect luciferase and sea pansy luciferase.

22. The method of claim 20, wherein the agent is a fluorophore.

23. The method of claim 22, wherein the fluorophore is a semiconductor nanocrystal.

24. The method of claim 23, wherein the fluorophore is a fluorescent dye.

25. The method of claim 20, wherein the agent is an enzyme, and a chemiluminescent substrate is used to detect the presence of agent.
